(19) United States
(12) Patent Application Publication
(10) Pub. No.: US 2022/0139530 A1
(43) Pub. Date: May 5, 2022

(54) SYSTEMS AND METHODS FOR PROCESSING MRI DATA

(71) Applicant: BlackThorn Therapeutics, Inc., San Francisco, CA (US)

(72) Inventors: Matthew KOLLADA, San Francisco, CA (US); Humberto Andres GONZALEZ CABEZAS, San Francisco, CA (US); Yuelu LIU, San Francisco, CA (US); Monika Sharma MELLEM, San Francisco, CA (US); Parvez AHAMMAD, San Francisco, CA (US); Qingzhu GAO, San Francisco, CA (US)

(21) Appl. No.: 17/594,234

(22) PCT Filed: Apr. 21, 2020
(86) PCT No.: PCT/US2020/029146

§ 371 Date: Oct. 7, 2021

Related U.S. Application Data
(60) Provisional application No. 62/841,420, filed on May 1, 2019.
(60) Provisional application No. 62/923,280, filed on Oct. 18, 2019.

(51) Int. Cl. G16H 30/20

(52) U.S. Cl. CPC G16H 30/20

(57) ABSTRACT

The present disclosure provides systems and methods for automating the QC of MRI scans. Particularly, the inventors trained machine learning classifiers using features derived from brain MR images and associated processing to predict the quality of those images, which is based on the ground truth of an expert's opinion. In one example, classifiers that utilized features derived from preprocessing log files (textual files output during MRI preprocessing) were particularly accurate and demonstrated an ability to be generalized to new datasets, which allows the disclosed technology to be scalable to new datasets and MRI preprocessing pipelines.

### CROSS-REFERENCE TO RELATED APPLICATIONS

This application claims priority to and the benefit of U.S. Provisional Patent Application No. 62/841,420, filed May 1, 2019, and U.S. Provisional Patent Application No. 62/923,280, filed Oct. 18, 2019, each of which is hereby incorporated by reference herein in its entirety.

### TECHNICAL FIELD

The present disclosure relates to processing MRI data.

### BACKGROUND

MRI data requires extensive preprocessing of the scanned images in order to construct a usable output dataset. Quality Control (QC) of MRI data processing is a substantial roadblock to analyzing large-scale datasets, and particularly affects the preprocessing features for fMRI data. Conventional data processing requires human involvement (e.g., “human-in-the-loop”). This human-involved data processing requires experts to manually identify correctly preprocessed output images. Often, the time requirement from expert reviewers is substantial.

Additionally, the preprocessing of structural and functional MRI scans is a computationally-intensive operation, typically taking several hours per subject (i.e., individual). This can result in prohibitively long waits between MRI data acquisition and analysis of the same, particularly in large datasets with many hundreds of subjects, especially when computation is performed using traditional computer infrastructure such as high-performance workstation units. The present disclosure is directed to solving these problems and addressing other needs.

### SUMMARY

According to some implementations of the present disclosure, systems and methods for automating the QC of MRI scans were developed. Particularly, machine learning classifiers were trained using features derived from brain MR images to predict the quality of those images, which is based on the ground truth of an expert's opinion. It is common practice in the field that expert QC reviewers examine raw MRI scans and pre-processed images to determine if the quality is sufficient for further analysis. The disclosed classifiers that are utilized to automate QC may incorporate a variety of features. In one example, classifiers that utilized features derived from preprocessing log files (textual files output during MRI preprocessing) were particularly accurate and demonstrated an ability to be generalized to new datasets, which also allows the disclosed technology to be scalable to new datasets and/or MRI preprocessing pipelines.

Additionally, in response to the limitations of conventional methods of processing and pre-processing MRI data, the present disclosure provides an automated search method for selecting optimal tMRI preprocessing pipeline parameters and automated methods of performing quality control. Implementations of the disclosed systems and methods have been validated on two independent datasets. The disclosed methods, for each subject (e.g., individual or patient), automatically searches a large set of preprocessing parameters to predict the particular preprocessing parameters that will allow scanned image of the subject to pass visual QC. Therefore, the disclosed systems and methods provide for generation of parameter set recommendations for each subject; these specific parameter sets dramatically reduce the turnaround time and effort required of an expert reviewer to fully quality control a dataset. The disclosed systems and methods therefore result in a novel, efficient, and effective technology to perform QC of preprocessed fMR images.

According to some implementations of the present disclosure, a method of analyzing MRI data provides for receiving unprocessed MRI data, corresponding to a set of MR images of a biological structure. The method then provides for preprocessing the received MRI data. Preprocessing includes (1) performing, for each MR image in the set of MR images, a structural-functional alignment and a skull-stripping procedure, and (2) outputting a plurality of parameter sets related to the preprocessing. The method then provides for generating a plurality of functional connectivity matrices (in some examples whole brain functional connectivity matrices) based on the plurality of parameter sets. The method then provides for identifying similar matrices in the plurality of functional connectivity matrices to yield a plurality of matrix clusters. The method then provides for selecting a dominant cluster of the plurality of matrix clusters. The method then provides for outputting a subset of parameters of the plurality of parameter sets corresponding to the dominant matrix.

In some examples, identifying similar matrices includes (1) determining a Frobenius norm of a pairwise difference between matrices in the plurality of functional connectivity matrices; (2) grouping matrices in the plurality of whole brain functional connectivity matrices into a subset cluster when the determined Frobenius norm is less than a threshold value; and (3) outputting the subset cluster into the plurality of matrix clusters.

In some examples, identifying similar matrices also includes increasing the threshold value until a size of a largest cluster in the plurality of matrix clusters is twice as large as a size of a next-largest cluster in the plurality of matrix clusters.

In some examples, the plurality of parameter sets corresponds to four parameters from a plurality of parameters associated with at least one of: the structural-functional alignment and skull-stripping procedure.

In some examples, the output subset of parameters corresponds to a centroid of the dominant cluster.

In some examples, the method further includes processing the received MRI data. with the output subset of parameters to yield a set of processed MR images.

In some examples, the received MRI data corresponds to MRI data for a subject. In some examples, the method further includes scanning a brain of a subject to output the set of MR images.

In some implementations, the present disclosure provides for a system including a memory and a control system. The memory contains a machine readable medium comprising machine executable code having stored thereon instructions for performing a method. The control system is coupled to the memory and includes one or more processors. The control system configured to execute the machine executable code to cause the control system to perform the method discussed above with respect to the disclosed method of analyzing MRI data. Additional examples of this system are as provided for above with respect to the disclosed method of analyzing MRI data.

In some implementations, the present disclosure provides for a non-transitory machine-readable medium. The medium has stored thereon instructions for performing a method and comprises machine executable code. The code, when executed by at least one machine, causes the machine to perform the disclosed method discussed above with respect to the disclosed method of analyzing MRI data. Additional examples of this non-transitory machine-readable medium are as provided for above with respect to the disclosed method of analyzing MRI data.

According to some implementations of the present disclosure, a system for analyzing MRI data includes a memory and a control system. The memory contains machine readable medium including machine executable code having stored thereon instructions for performing a method. The control system is the memory. The control system has one or more processors. The control system is configured to execute the machine executable code to cause the control system to receive unprocessed MRI data corresponding to a set of MR images. A preprocessing is performed on the received unprocessed MRI data to output a preprocessed set of MR images. A set of features related to the preprocessing is outputted. Using a machine learning model, the set of features is processed to determine a subset of the preprocessed set of MR images that have a threshold image quality.

In some examples, the threshold image quality includes an image quality sufficient to pass manual quality control.

In some examples, the threshold image quality includes an image quality suitable for further processing by a model to identify a set of functional Magnetic Resonance Imaging (fMRI) features. In some such implementations, the set of fMRI features includes at least functional connectivity.

In some examples, the preprocessing includes performing, for each MR image in the set of MR images, a structural-functional alignment.

In some examples, the machine learning model includes a logistic regression model, a support vector machine, a gradient boosting machine, or a random forest model.

In some examples, the machine learning model is trained using outcome labels based on manual QC ratings.

In some examples, the set of features includes a set of log data from Mill preprocessing runtime logs. In some such examples, the set of log data from MRI preprocessing runtime logs includes data in text format relating to a quantitative assessment of structural-functional alignment, in some other such examples, the set of log data from MRI preprocessing runtime logs includes at least one of: preprocessing step runtimes, brain coordinates, structural-functional alignment cost values, a quantity of edits made to the set of MR images, and an angle of image capture of the brain in the set of MR images.

In some examples, the control system is further configured to store the subset of the set of MR images in the memory.

In some examples, the preprocessing further includes a skull stripping procedure.

In some examples, the preprocessed set of MR images includes structural MR images.

In some examples, the preprocessed set of MR images includes functional MR images.

In some examples, the set of MR images includes unprocessed functional MRI data and unprocessed structural MRI data representing a brain for each patient.

According to some implementations of the present disclosure, a method for analyzing MRI data includes receiving unprocessed MRI data corresponding to a set of MR images. A preprocessing is performed on the received unprocessed MRI data to output a preprocessed set of MR images. A set of features related to the preprocessing is outputted. Using a machine learning model, the set of features is processed to determine a subset of the preprocessed set of MR images that have a threshold image quality.

According to some implementations of the present disclosure, a non-transitory machine-readable medium has stored thereon instructions for performing a method. The non-transitory machine-readable medium includes machine executable code, which when executed by at least one machine, causes the machine to analyze MRI data includes receiving unprocessed MRI data corresponding to a set of MR images. A preprocessing is performed on the received unprocessed MRI data to output a preprocessed set of MR images. A set of features related to the preprocessing is outputted. Using a machine learning model, the set of features is processed to determine a subset of the preprocessed set of MR images that have a threshold image quality.

In some implementations, a method of analyzing MRI data includes first receiving unprocessed MRI data. The unprocessed MRI data includes a plurality of sets of MR images of a biological structure. Each set of MR images corresponds to a patient in a plurality of patients. The method then provides for preprocessing the received MRI data. The preprocessing includes parallel processing of sequential images in each set of MR images. The method then provides for outputting parcelated and voxel-level pre-processed time series for each set of MR images, based on the preprocessing of the received MRI data.

In some examples, the unprocessed MRI data comprises raw structural MRI data and raw resting-state functional MRI data.

In some examples, preprocessing the received MRI data includes performing a series of preprocessing steps. The series of preprocessing steps includes at least one of: structural preprocessing, despiking, motion correction, skull-stripping, co-registration between structural and functional images, spatial smoothing, normalization by mean signal, nuisance signal regression, and normalization to Talairach coordinates. The steps can be performed in any order.

In some examples, preprocessing the received MRI data includes performing, for each MR image in each set of MR images, (1) a structural-functional alignment, and (2) a skull-stripping procedure. The method can then provide for outputting a plurality of parameter sets related to the preprocessing. The method can then provide for generating a plurality of functional connectivity matrices based on the plurality of parameter sets; identifying similar matrices in the plurality of functional connectivity matrices to yield a plurality of matrix clusters; selecting a dominant cluster of the plurality f matrix clusters; and outputting a subset of parameters of the plurality of parameter sets corresponding to the dominant matrix. This can be performed in accordance with method 200 of FIG. 2, as discussed above.

In some examples of the above preprocessing, identifying similar matrices includes (1) determining a Frobenius norm of a pairwise difference between matrices in the plurality of functional connectivity matrices; (2) grouping matrices in the plurality of functional connectivity matrices into a subset cluster when the determined Frobenius norm is less than a threshold value; and (3) outputting the subset cluster into the plurality of matrix clusters. In some examples, the method can then provide for increasing the threshold value until a size of a largest cluster in the plurality of matrix clusters is twice as large as a size of a next-largest cluster in the plurality of matrix clusters. In some examples, the plurality of parameter sets corresponds to four parameters from a plurality of parameters associated with at least one of: the structural-functional alignment and skull-stripping procedure. In some examples, the output subset of parameters corresponds to a centroid of the dominant cluster. In some examples, the method can further provide for preprocessing each set of images in the plurality of sets of MR images, based on the output subset of parameters.

In some examples, each set of MR images corresponds to MRI data of a biological structure of a subject.

In some examples, the method further provides for scanning a brain of a subject to output the set of MR images.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1 shows a system for performing methods of pre-processing MRI data, according to some implementations of the present disclosure.

FIG. 2 shows a method for pre-processing MRI data, according to some implementations of the present disclosure.

FIG. 3 is a block diagram of an MRI system used to acquire NMR data, according to some implementations of the present disclosure.

FIG. 4 is a block diagram of a transceiver which forms part of the MRI system of FIG. 3, according to some implementations of the present disclosure.

FIG. 5 shows a method for automating quality control (“QC”) processes of MRI data, according to some implementations of the present disclosure.

FIGS. 6A-6C are graphs showing the performance of various machine learning models for automated QC, according to some implementations of the present disclosure.

FIG. 7 shows a method for automating quality control (“QC”) processes of MRI data, according to some implementations of the present disclosure.

FIG. 8 illustrates example preprocessed images that have passed and filed QC, according to some implementations of the present disclosure.

FIG. 9 illustrates a flow chart showing examples of preprocessing pipelines, according to some implementations of the present disclosure.

FIG. 10 shows an example excerpt from a preprocessing log, according to some implementations of the present disclosure.

FIGS. 11A-11D illustrate graphs showing the performance of various machine learning models for automated QC, according to some implementations of the disclosure. FIG. 12A illustrates the performance using the FLAG-QC features; FIG. 11B illustrates the performance of all features; FIG. 11C illustrates the performance of MRIQC features for structural MRI; and FIG. 11D illustrates the performance of MRIQC features for functional MRI.

FIGS. 12A-12D illustrate graphs showing the performance of various machine learning models for automated QC, according to some implementations of the disclosure. FIG. 12A illustrates the performance using the FLAG-QC features using random forest; FIG. 12B illustrates the performance of all features using random forest; FIG. 12C illustrates the performance of MRIQC features for structural MRI using a gradient boosting machine; and FIG. 11D illustrates the performance of MRIQC features for functional MRI using logistic regression.

While the present disclosure is susceptible to various modifications and alternative forms, specific implementations have been shown by way of example in the drawings and will be described in further detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

The present invention is described with reference to the attached figures, where like reference numerals are used throughout the figures to designate similar or equivalent elements. The figures are not drawn to scale, and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a method in accordance with the present invention.

### Overview

Raw fMR images must undergo a complex set of computational transformations, often termed preprocessing, before being used in any statistical analysis. These raw and preprocessed images are commonly manually assessed for quality by expert reviewers in a process referred to as “quality control” (QC). These reviewers, often in multiple steps, visualize the preprocessed images, and inspect them for apparent errors that may erroneously bias future analysis. Many evaluation schemes for QC have been proposed. However, there exists a need for one simple, clear strategy to determine whether the scan (i) passes and is therefore usable, or (ii) fails and is discarded from further analysis.

The labor-intensive and/or time-consuming nature of QC can be a bottleneck to the analysis of fMR images at scale. QC of an fMRI dataset with hundreds of scans can take weeks to months of manual assessment from a single expert reviewer before analysis can begin. As discussed herein, many recent fMRI studies have collected data at or even above that scale, providing compelling motivation for the field to develop a scalable QC framework to (i) reduce the burden on individual researchers, and (ii) standardize quality control of fMRI data.

Accordingly, the systems and methods are disclosed for automating the QC of MRI scans. For example, machine learning classifiers can be trained using features derived from brain MR images to predict the quality of those images, which is based on the ground truth of an expert's opinion. Conventionally, expert QC reviewers examine raw MRI scans and preprocessed images to determine if the quality is sufficient for further analysis. The disclosed classifiers are utilized to automate QC, and can incorporate a variety of features. In some implementations, classifiers that utilized features derived from preprocessing log files (e.g., textual files output during MRI preprocessing) were found particularly accurate, and further demonstrated its ability to be generalized to new datasets, which also allows the disclosed technology to be scalable to new datasets and/or MRI preprocessing pipelines.

Additionally, in response to the limitations of conventional methods of processing and pre-processing MRI data, the present disclosure provides (i) an automated search method for selecting optimal fMRI preprocessing pipeline parameters, (ii) automated methods of QC, and associated systems and methods. Implementations of the disclosed systems and methods have been validated on two independent datasets. Some of the disclosed systems and methods, automatically searches a large set of preprocessing parameters for each subject, to predict the particular preprocessing parameters that will allow scanned image of the subject to pass visual QC. Therefore, the disclosed systems and methods provide for generation of parameter set recommendations for each subject; these specific parameter sets dramatically reduce the turnaround time and effort required of an expert reviewer to fully quality control (QC) a dataset. The disclosed systems and methods therefore results in a novel, efficient, and effective method to perform QC of preprocessed fMR images.

### Systems

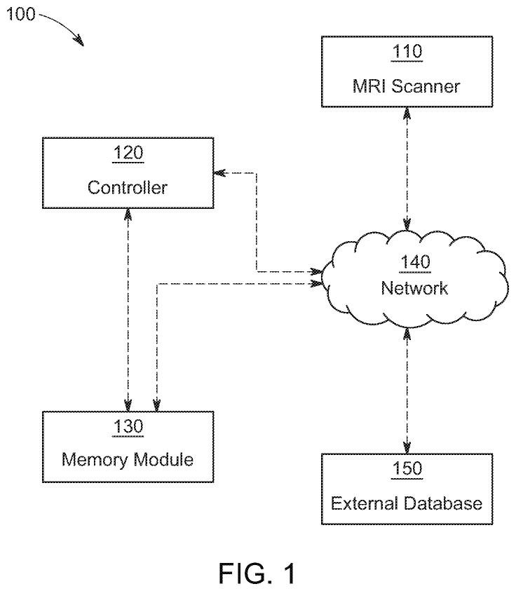

FIG. 1 shows a system 100 for performing methods of pre-processing data and/or QC MRI datasets, according to some implementations of the present disclosure. System 100 includes an MRI scanner 110, a controller 120, a memory module 130, a network 140, and an external database 150. The MRI scanner 110 scans biological structures of one or more subjects (e.g., individuals, patients). The MRI scanner 110 can send scanned images corresponding to the biological structures to the external database 150 via the network 140 and/or to the memory module 130. In some implementations, the MRI scanner 110 can send a plurality of scanned images corresponding to a particular patient.

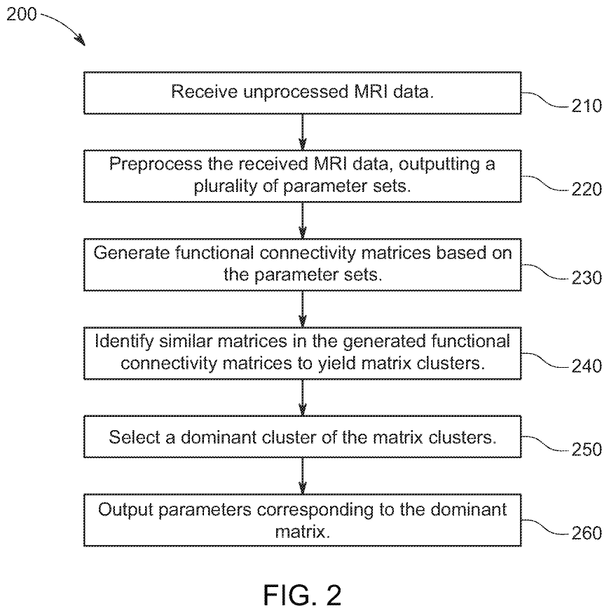

In some implementations, the MRI scanner 110 can be controlled by an external computing device through the network 140. For example, the external computing device can include the controller 120 and the memory module 130. In some implementations, the external computing device includes the external database 150, and/or has access to the external database 150. In some implementations, the controller 120 processes scanned images from the MRI scanner 110 in accordance with the method 200 of FIG. 2, as discussed further herein. In some implementations, the external database 150 includes a storage device for a plurality of user data (e.g., patient data). The user data can include MRI scans captured by the MRI scanner 110, and/or any other health data as known in the art.

### Example Method of Parameter Selection

In some instances, the parameters utilized to control an MRI scanner (e.g., the MRI scanner 110 of the system 100) during data acquisition may impact the quality and characteristics of the resulting images. Accordingly, in some implementations, methods are discussed for selecting optimal parameters for MR image acquisition. For example, FIG. 2 shows a method for pre-processing MRI data to select optimal parameters, according to some implementations of the present disclosure. In other example methods disclosed herein, the parameters may be standard and/or predefined parameters, used for each scan in a study.

In some implementations, the method 200 begins at step 210 by receiving unprocessed MRI data. In some examples, the unprocessed MRI data corresponds to a set of MR images of a biological structure. The biological structure can be a subject's (e.g., a patient's) brain. The received MRI data can correspond to any type of MRI data for a subject. In some examples, the method 200 starts with scanning a brain of a subject to output the set of MR images.

Step 220 of the method 200 then provides for preprocessing the received MRI data. Preprocessing the data includes performing, for each MR image in the set of MR images, a structural-functional alignment and a skull-stripping procedure. In some implementations, step 220 further provides for outputting a plurality of parameter sets related to the preprocessing.

Step 230 of the method 200 provides for generating a plurality of functional connectivity matrices based on the plurality of parameter sets output in step 220. In some examples, the plurality of functional connectivity matrices may include whole brain functional connectivity matrices.

Step 240 of the method 200 provides for identifying similar matrices in the plurality of functional connectivity matrices and/or whole brain functional connectivity matrices. In some implementations, the identified similar matrices are grouped to yield a plurality of matrix clusters.

In some implementations, identifying similar matrices includes (1) determining a Frobenius norm of a pairwise difference between matrices in the plurality of whole brain functional connectivity matrices; (2) grouping matrices in the plurality of whole brain functional connectivity matrices into a subset cluster when the determined Frobenius norm is less than a threshold value; and/or (3) outputting the subset cluster into the plurality of matrix clusters.

In some implementations, the threshold value can be increased until a size of a largest cluster in the plurality of matrix clusters is twice as large as a size of a next-largest cluster in the plurality of matrix clusters. In some implementations , the plurality of parameter sets corresponds to four parameters from a plurality of parameters associated with at least one of: the structural-functional alignment and skull-stripping procedure.

Step 250 of the method 200 provides for selecting a dominant cluster of the plurality of matrix clusters. Step 260 of the method 200 provides for outputting a subset of parameters of the plurality of parameter sets corresponding to the dominant matrix. In some implementations, the output subset of parameters corresponds to a centroid of the dominant cluster.

In some implementations, the method 200 further includes processing the received MRI data with the output subset of parameters to yield a set of processed MR images.

### Example NMR Systems

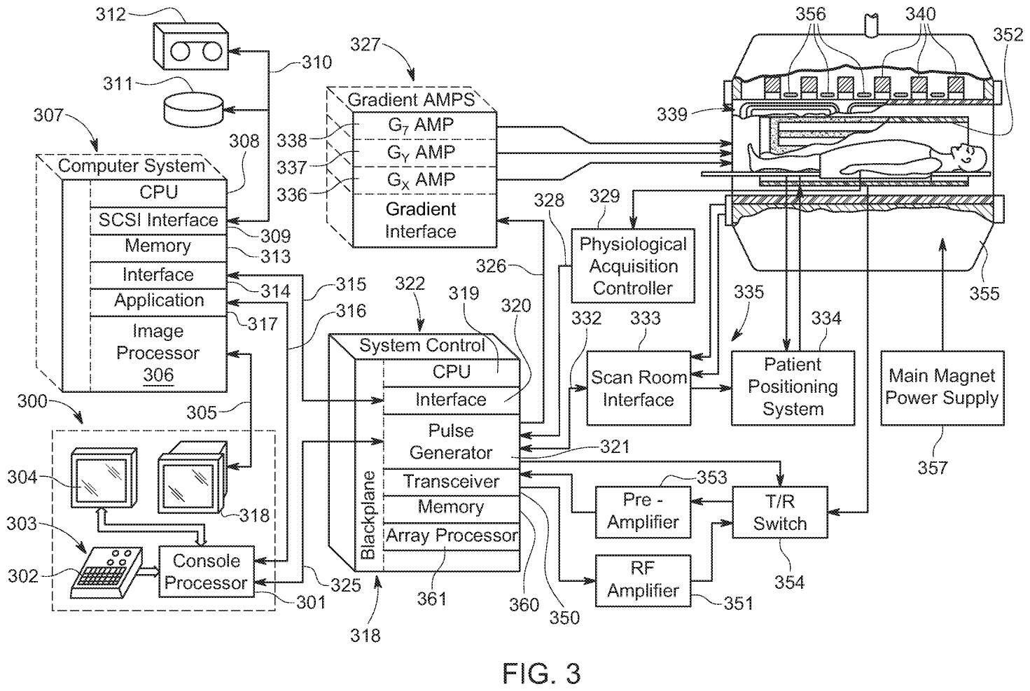

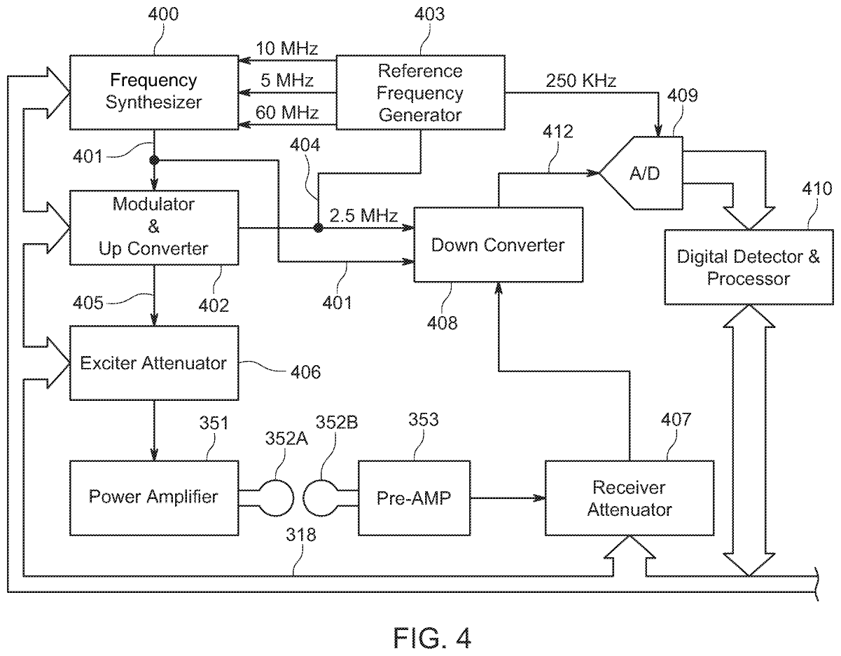

Referring generally to FIG. 3, the systems and methods of the present disclosure can, alternatively or additionally, be performed on a nuclear magnetic resonance (NMR) system. In some implementations, NMR can include the hardware used to generate different types of scans, including MRI scans. Referring generally to FIGS. 3 and 4, as shown, an example of the major components of an NMR system can be used to carry out the systems and methods of the various implementations disclosed herein. FIG. 4 illustrates the components of a transceiver for the NMR system of FIG. 3. It should be noted that the systems and methods of the various implementations of the present disclosure can also be carried out using other NMR systems and/or other settings, ranges, or components.

The operation of the system illustrated in FIGS. 3 and 4 is controlled from an operator console 300, which includes a console processor 301 that scans a keyboard 302. In some implementations, the operator console 300 receives inputs from a human operator through, for example, a control panel 303 and/or a plasma display/touch screen 304. The console processor 301 communicates through a communications link 316 with an applications interface module 317 in a separate computer system 307. Through the keyboard 302 and the controls 303, an operator controls the production and display of images by an image processor 306 in the computer system 307. In some implementations, the image processor 306 connects directly to a video display 318 on the console 300 through a video cable 305.

The computer system 307 is formed about a backplane bus which conforms with the VME standards, and includes a number of modules that communicate with each other through this backplane. In addition to the application interface 317 and the image processor 306, the computer system 307 can further include a CPU module 308 that controls the VME backplane, and/or an SCSI interface module 309 that connects the computer system 307 through a bus 310 to a set of peripheral devices (e.g., the disk storage 311, and the tape drive 312). In some implementations, the computer system 307 also includes a memory module 313 (e.g., as a frame buffer for storing image data arrays), and/or a serial interface module 314 that links the computer system 307, through a high speed serial link 315, to a system interface module 320 located in a separate system control cabinet 322.

In some implementations, the system control 322 includes a series of modules, which are connected together by a common backplane 318. The backplane 318 includes a. number of bus structures, such as a bus structure controlled by the CPU module 319. The serial interface module 320 connects this backplane 318 to the high speed serial link 315, and pulse generator module 321 connects the backplane 318 to the operator console 300 through a serial link 325. It is through this link 325 that the system control 322 receives commands from the operator which indicate the scan sequence that is to be performed.

The pulse generator module 321 operates the system components to carry out the desired scan sequence. The pulse generator module produces data which indicates the timing, strength and shape of the RF pulses which are to be produced, and the timing of and length of the data acquisition window. The pulse generator module 321 also connects through serial link 326 to a set of gradient amplifiers 327, and conveys data thereto which indicates the timing and shape of the gradient pulses that are to be produced during the scan. The pulse generator module 321 also receives user data through a serial link 328 from a physiological acquisition controller 329.

The physiological acquisition control 329 can receive a signal from a number of different sensors connected to the patient. For example, it may receive ECG signals from electrodes or respiratory signals from a bellows and produce pulses for the pulse generator module 321 that synchronizes the scan with the patient's cardiac cycle and/or respiratory cycle. And finally, the pulse generator module 321 connects through a serial link 332 to scan room interface circuit 333, which receives signals at inputs 335 from various sensors associated with the position and condition of the patient and the magnet system. It is also through the scan room interface circuit 333 that a patient positioning system 334 receives commands, which move the patient cradle and transport the patient to the desired position for the scan.

The gradient waveforms produced by the pulse generator module 321 are applied to a gradient amplifier system 327 comprised of Gx, Gy, and Gz amplifiers 336, 337 and 338, respectively. Each amplifier 336, 337, and 338 is utilized to excite a corresponding gradient coil in an assembly generally designated 339. The gradient coil assembly 339 forms part of a magnet assembly 355, which includes a polarizing magnet 340 that produces a 1.5 Tesla polarizing field that extends horizontally through a bore.

The gradient coils 339 encircle the bore. When energized, the gradient coils 339 generate magnetic fields in the same direction as the main polarizing magnetic field, but with gradients Gx, Gy and Gz directed in the orthogonal x-, y- and z-axis directions of a Cartesian coordinate system. That is, if the magnetic field generated by the main magnet 440 is directed in the z direction and is termed BO, and the total magnetic field in the z direction is referred to as Bz, then Gx∂Bz/∂x, Gy=∂Bz/∂y and Gz=∂Bz/∂z, and the magnetic field at any point (x,y,z) in the bore of the magnet assembly 441 is given by B(x,y,z)=Bo+Gxx+GyyGzz.

The gradient magnetic fields are utilized to encode spatial information into the NMR signals emanating from the patient being scanned. Because the gradient fields are switched at a very high speed when an EPI sequence is used to practice some implementations of the present disclosure, local gradient coils are employed in place of the whole-body gradient coils 139. These local gradient coils are designed for the head and are in close proximity thereto. This enables the inductance of the local gradient coils to be reduced and the gradient switching rates increased as required for the EPI pulse sequence. Examples of local gradient coils include what is disclosed in U.S. Pat. No. 5,372,137, issued on Dec. 13, 1994 and entitled “NMR Local Coil For Brain Imaging,” which is incorporated herein by reference.

Located within the bore 342 is a circular cylindrical whole-body RF coil 352. This coil 352 produces a circularly polarized RF field in response to RF pulses provided by a transceiver module 350 in the system control cabinet 322. These pulses are amplified by an RF amplifier 351 and coupled to the RF coil 352 by a transmit/receive switch 354, which forms an integral part of the RF coil assembly. Waveforms and/or control signals are provided by the pulse generator module 321, and utilized by the transceiver module 350 for RF carrier modulation and mode control. The resulting NMR signals radiated by the excited nuclei in the patient may be sensed by the same RF coil 352, and coupled through the transmit/receive switch 354 to a preamplifier 353. In some implementations, the amplified NMR signals are demodulated, filtered, and digitized in the receiver section of the transceiver 350.

The transmit/receive switch 354 is controlled by a signal from the pulse generator module 321 to electrically connect the RF amplifier 351 to the coil 352 during the transmit mode, and to connect the preamplifier 353 during the receive mode. The transmit/receive switch 354 also enables a separate local RF head coil to be used in the transmit and receive mode to improve the signal-to-noise ratio of the received NMR signals. With NMR systems, a local RF coil is preferred in order to detect small variations in NMR signal. Examples of local RF coil includes the local RF coil disclosed in the above-cited U.S. Pat. No, 5,372,137, which is incorporated herein by reference.

In addition to supporting the polarizing magnet 340, the gradient coils 339, and RF coil 352, the main magnet assembly 341 also supports a set of shim coils 356 associated with the main magnet 340 and used to correct inhomogeneities in the polarizing magnet field. The main power supply 357 is utilized to bring the polarizing field produced by the superconductive main magnet 340 to the proper operating strength and is then removed.

The NMR signals picked up by the RF coil are digitized by the transceiver module 350, and transferred to a memory module 360, which is also part of the system control 322. When the scan is completed and an entire array of data has been acquired in the memory modules 360, an array processor 361 operates to Fourier transform the data into an array of image data. This image data is conveyed through the serial link 315 to the computer system 307 where it is stored in the disk memory 311. In response to commands received from the operator console 300, this image data may be archived on the tape drive 312, or it may be further processed by the image processor 1306 and conveyed to the operator console 300 and presented on the video display 318 as will be described in more detail hereinafter.

Referring particularly to FIG. 4, the transceiver 350 (FIG. 3) includes components that produce the RE excitation field B1 through power amplifier 351 at a coil 352A and components which receive the resulting NMR signal induced in a coil 352B. Similar to the coil 352 (FIG. 3) discussed above, the coils 352A and 352B may be a single whole-body coil. However, the best results are achieved with a single local RF coil specially designed for the head. The base, or carrier, frequency of the RE excitation field is produced under control of a frequency synthesizer 400, which receives a set of digital signals (CF) through the backplane 318 from the CPU module 319 (FIG. 3) and pulse generator module 321 (FIG. 3). These digital signals indicate the frequency and phase of the RE carrier signal, which is produced at an output 401.

The commanded RF carrier is applied to a modulator and up converter 402 where its amplitude is modulated in response to a signal R(t) also received through the backplane 318 from the pulse generator module 321. The signal R(t) defines the envelope, and therefore the bandwidth, of the RF excitation pulse to be produced. It is produced in the module 321 by sequentially reading out a series of stored digital values that represent the desired envelope. These stored digital values may, in turn, be changed from the operator console 300 (FIG. 3) to enable any desired RF pulse envelope to be produced.

The modulator and up converter 402 produces an RF pulse at the desired Larmor frequency at an output 405. The magnitude of the RF excitation pulse output through line 405 is attenuated by an exciter attenuator circuit 406 which receives a digital command, TA, from the backplane 318. The attenuated RF excitation pulses are applied to the power amplifier 351 that drives the RE coil 352A. Examples of this portion of the transceiver 322 includes what is disclosed in U.S. Pat. No. 4,952,877, which is incorporated herein by reference.

Referring still to FIGS. 3 and 4, the NMR signal produced by the subject is picked up by the receiver coil 352B, and applied through the preamplifier 353 to the input of a receiver attenuator 407. The receiver attenuator 407 further amplifies the NMR signal; and this is attenuated by an amount determined by a digital attenuation signal (RA) received from the backplane 318. The receive attenuator 407 is also turned on and off by a signal from the pulse generator module 321 such that it is not overloaded during RE excitation.

The received NMR signal is at or around the Larmor frequency, which in some implementations is around 63.86 MHz for 1.5 Tesla. This high frequency signal is down converted in a two-step process by a down converter 408, which first mixes the NMR signal with the carrier signal on line 401, and then mixes the resulting difference signal with the 2.5 MHz reference signal on line 404. The resulting down converted NMR signal on line 412 has a maximum bandwidth of 125 kHz and it is centered at a frequency of 187.5 kHz.

The down converted NMR signal is applied to the input of an analog-to-digital (A/D) converter 409 which samples and digitizes the analog signal at a rate of 250 kHz. The output of the AID converter 409 is applied to a digital detector and signal processor 410, which produce 16-bit in-phase (1) values and 16-bit quadrature values (Q values) corresponding to the received digital signal. The resulting stream of digitized I and Q values of the received NMR signal is output through backplane 318 to the memory module 360 where they are employed to reconstruct an image.

To preserve the phase information contained in the received NMR signal, both the modulator and up converter 402 in the exciter section and the down converter 408 in the receiver section are operated with common signals. More particularly, the carrier signal at the output 401 of the frequency synthesizer 400, and the 2.5 MHz reference signal at the output 404 of the reference frequency generator 403 are employed in both frequency conversion processes. Phase consistency is thus maintained and phase changes in the detected NMR signal accurately indicate phase changes produced by the excited spins. The 2.5 MHz reference signal as well as 5, 10, and 60 MHz reference signals are produced by the reference frequency generator 403 from a common 20 MHz master clock signal. The latter three reference signals are employed by the frequency synthesizer 400 to produce the carrier signal on output 401. Examples of the receiver includes what is disclosed in U.S. Pat. No. 4,992,736, which is incorporated herein by reference.

### EXAMPLE 1

### Parameter Selection

In response to the limitations of conventional systems and methods of processing and/or pre-processing MRI data, the present disclosure provides an automated search method for selecting optimal fMRI preprocessing pipeline parameters. Implementations of the disclosed systems and methods have been validated on two independent datasets.

For example, MRI data was preprocessed from two publicly available MRI datasets, CNP LA5c1 (N=251) and EMBARC2 (N=330), using 72 different parameter sets. This was enabled by the disclosed technologies' ability to perform parallel fMRI preprocessing in a massive scale and with a cloud-enabled pipeline based on AFNI. These 72 parameter sets were created by varying four different parameters that commonly need human-led optimization—two from the structural-functional alignment step and two from the skull stripping step.

For each of the 72 pipeline outputs per subject, whole brain Functional Connectivity (IV) matrices were generated and grouped by similarity into clusters based on the Frobenius norm of the pairwise difference between the matrices. The similarity threshold used to group the matrices was set as the smallest value such that a dominant, stable cluster was found, indicated by the size ratio between the two largest clusters being at least 2 to 1. The centroid of the largest cluster of parameters for each subject was selected as our prediction to pass QC and the algorithm-generated predictions were validated using visual QC from expert reviewers.

The automatic parameter prediction method was compared to a control method of using a single, expert-selected set of parameters for subjects in two independent datasets. The control method was chosen as an estimate of results given the same amount of reviewer effort without our prediction method. Using 50 randomly selected subjects from each dataset, the automatic parameter prediction method had 92% of subjects pass visual QC for CNP and 80% for EMBARC, while the control method passed only 62% of subjects for CNP and 70% for EMBARC.

### EXAMPLE 2

### Parallel Processing for QC for Parameter Selection

In some implementations of the present disclosure, preprocessing the received MRI data can include parallel processing. Preprocessing of structural and functional MRI scans is a computationally-intensive operation, typically taking several hours per subject. This results in prohibitively long waits between MRI data acquisition and analysis, particularly in large datasets with many hundreds of subjects, and especially when computation is performed using traditional computer infrastructure such as high-performance workstation units.

The present disclosure provide for a cloud-enabled and/or massively-parallel NMI preprocessing pipeline. The parallel pre-processing can include any suitable parallel processing technologies. In some implementations, the method provides for preprocessing an average of more than 150 scans per day. For example, in some implementations, a preprocessing pipeline can be built using FreeSurfer and NEM software suites. The pipeline can take raw structural and/or resting-state functional MRI data, and output parcelated and/or voxel-level preprocessed time series as well as functional connectivity matrices.

In some implementations, several steps can be taken to preprocess the raw data before using the pipeline. These steps include: structural preprocessing, despiking, motion correction, skull-stripping, co-registration between structural and functional images, spatial smoothing, normalization by mean signal, nuisance signal regression, normalization to the MNI space, or the like, or any combination thereof. The disclosed pipeline follows the Brain Imaging Data Structure (BIDS) standard and can be used as a cloud service; which includes retrieving and storing files on demand in AWS S3 and executing in Docker containers that require minimal support. The disclosed pipeline is also compatible with AWS Batch, enabling the preprocessing of complete datasets in parallel using a cloud-based cluster environment.

In one experimental implementation of the disclosed pipeline, resting-state scans from the following datasets were preprocessed: ABIDE I, CNP, and EMBARC. The disclosed pipeline preprocessed the CNP dataset in 43 hours (N=251, 5.8 subjects/hour); the EMBARC dataset in 42 hours (N=326, 7.7 subjects/hour); and the ABIDE I dataset in 80 hours (N=1056, 13.2 subjects/hour). The containerized pipeline code was executed in “c5” AWS EC2 computers with a limit of 8 GB of RAM per container. These results were obtained with a limit of using up to 1300 concurrent AWS EC2 vCPUs.

Therefore, the disclosed MRI preprocessing pipeline is a step forward in bringing state-of-the-art technology to neuro-imaging analysis by creating a flexible on-demand high-performance computing infrastructure with minimal offline footprint and long-term cost. Importantly, the significant reduction in end-to-end preprocessing time for complete MRI datasets enables scientists to study the effect and sensitivity of parameter changes and opens the door for big data (datasets with many thousands of subjects) analysis among MRI datasets.

### EXAMPLE 3

### Machine Learning Based Automated QC

Over the last twenty-five (25) years, advances in the collection and analysis of functional magnetic resonance imaging (fMRI) data have enabled new insights into the brain basis of human health and disease. Individual behavioral variation can now be visualized at a neural level as patterns of connectivity among brain regions. As such, functional brain imaging is enhancing our understanding of clinical psychiatric disorders by revealing ties between regional and network abnormalities and psychiatric symptoms.

Initial success in this arena has recently motivated collection of larger datasets, which are needed to leverage fMRI to generate brain-based biomarkers to support the development of precision medicines. Despite methodological advances and enhanced computational power, evaluating the quality of fMRI scans remains a critical step in the analytical framework. Before analysis can be performed, expert reviewers visually inspect individual raw scans and preprocessed derivatives to determine viability of the data. This QC process is labor intensive, and the inability to adequately automate at large scale has proven to be a limiting factor in clinical neuroscience.

For example, raw fMR images must undergo a complex set of computational transformations, often termed preprocessing, before being used in any statistical analysis. These raw and preprocessed images are commonly manually assessed for quality by expert reviewers in a process referred to as quality control/QC. These reviewers, often in multiple steps, visualize the preprocessed images, and inspect them for apparent errors that may erroneously bias future analysis. Many evaluation schemes for QC have been proposed. However, there still exists a need for one simple, clear strategy to determine whether the scan passes and is therefore usable, or fails and is discarded from further analysis. The present disclosure thus addresses this need and others.

The labor-intensive and time-consuming nature of QC is bottleneck to the analysis of fMR images at scale. QC of an fMRI dataset with hundreds of scans, which can take weeks to months of manual assessment from a single expert reviewer before analysis can begin. As discussed herein, many recent fMRI studies have collected data at or even above that scale, providing compelling motivation to develop a scalable QC framework to reduce the burden on individual researchers and standardize quality control of ATM data. The present disclosure thus provides this scalable QC framework.

Accordingly, technology for automating the QC of MR scans is disclosure. For example, in some implementations, machine learning classifiers are trained using features derived from brain MR images to predict the quality of those images, which is based on the ground truth of an expert's opinion. Typically, expert QC reviewers examine raw MRI scans and pre-processed images to determine if the quality is sufficient for further analysis. For volumetric data, the 3D preprocessed MR images are spatially sampled as 2D images for easier assessment by the reviewer.

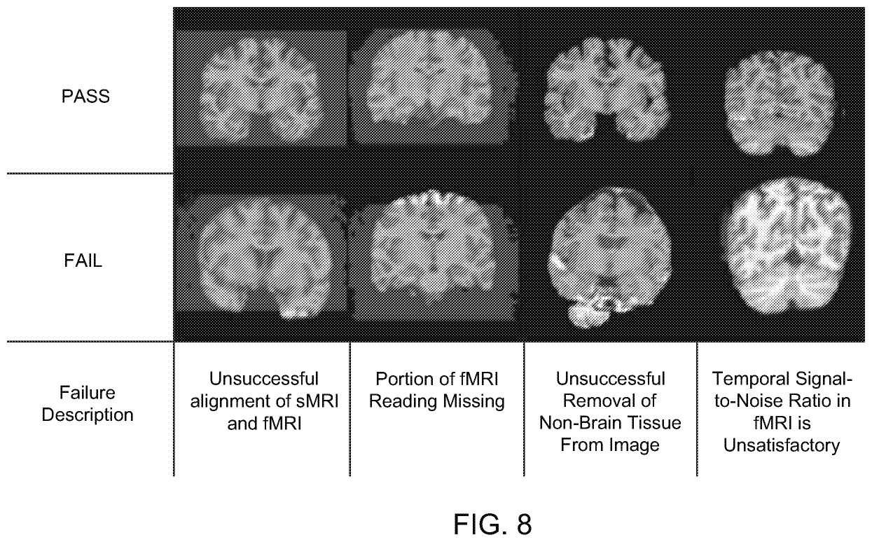

Referring to FIG. 8, examples of 2D images that “pass” and “fail” QC are shown with common failure points, such as misalignment of structural and functional MRI scans or unsuccessful automatic removal of non-brain tissue. In some examples, following assessment of image quality of raw data and across the multiple preprocessing steps, the reviewer made a binary “pass” or “fail” decision for each subject's fMRI scan. Thus, an fMRI scan is tagged as useable (pass) or not (fail), and these labels serve as the ground-truth decisions on which the disclosed classifiers are trained.

The classifiers were tested on data collected from additional studies (e.g., different than those used to train the classifies). The predictions using the classifiers were able to be generalized across data from different studies. This is particularly important, because previous attempts to automate QC generalized poorly. Furthermore, no known attempts have been made to apply an automated QC framework to fMRI data.

In addition, the automatic QC classifiers were applied to two large, open-source fMRI datasets. The classifiers were used to evaluate a range of feature sets, including one entitled “FMRI preprocessing Log mining for Automated, Generalizable Quality Control” (FLAG-QC). Specifically, the ability of these classifiers to generalize across fMRI data. collected within different studies was evaluated. The results demonstrated that the classifiers were able to achieve this generalization using only the novel FLAG-QC feature set proposed within this disclosure—the log based features discussed herein.

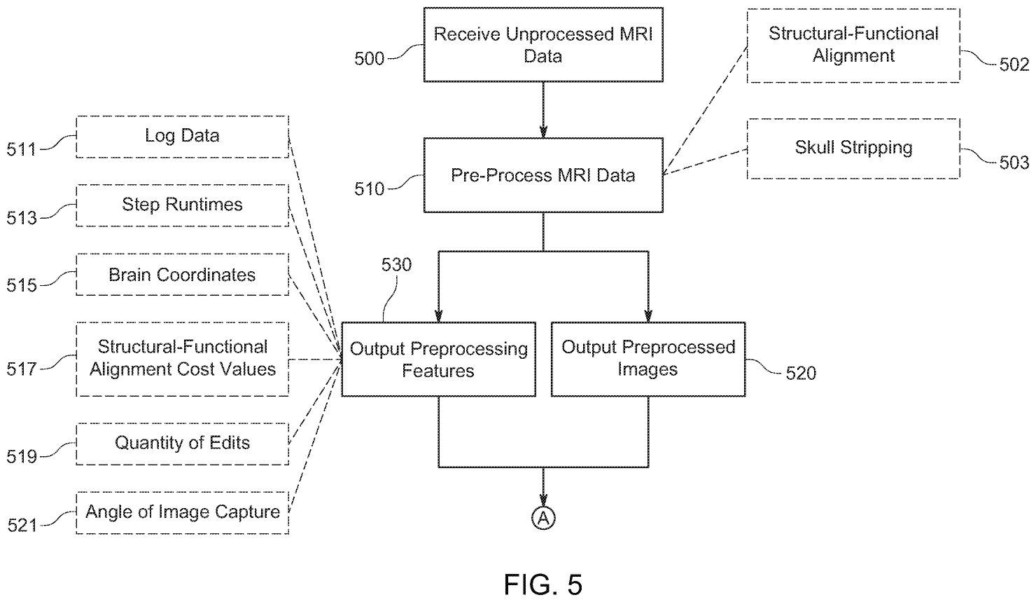

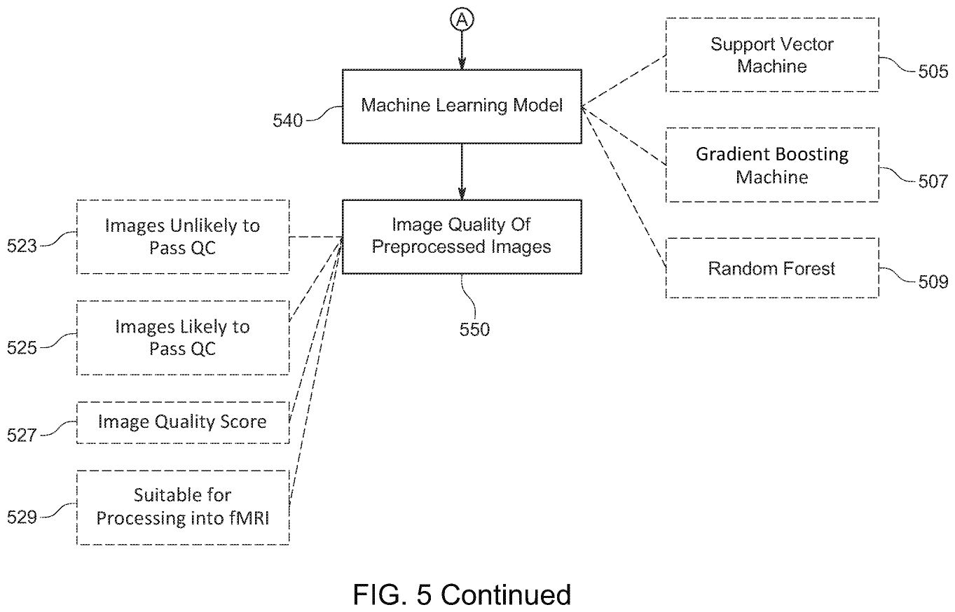

Referring now to FIG. 5, a flow chart is illustrated and shows an example of a method for predicting which images of a set of MR images will pass quality control. The method may utilize certain parameters generated as a result of the preprocessing methods disclosed herein as input parameters to a machine learning model for each of the images. In other implementations, the method may utilize standard parameters to process the MRI data.

First, raw, unprocessed MR data may be received (step 500) that is, for example, output from a scanner and/or stored in a database. Then, the raw MR data may be pre-processed (step 510), for instance, into images. This may include various steps based on the types of images that are being created, including skull stripping steps 503 and/or structural-functional alignment steps 502, if the images are functional magnetic resonance images (fMRI). During the preprocessing steps. various features may he output (step 530) that are a result or created during preprocessing.

These features may include log data 511, runtimes of various steps of preprocessing 513, brain coordinates 515, cost or error values associated with structural-functional alignment 517, quantity of edits made to the images 519, angle of image capture 521, or others, or a combination thereof. Then, the preprocessed images (from step 520) and/or the preprocessing features (from step 530), or other features may be input into a machine learning model 540 to output an image quality of the preprocessed images 550.

The machine learning model 540 can include a support vector machine 505, a gradient boosting machine 507, random forest 509, or other suitable machine learning model, or any combination therefore. In some implementations, the machine learning model 540 utilized includes a classification of pass 523 or fail 525 for the output preprocessed images 520, and/or whether it is suitable for processing into fMR images. In some implementations, the machine learning model 540 may output a quantitative assessment of the image quality of the preprocessed images, such as an image quality score 527. In some implementations, the machine learning model 540 may be trained with data using manual QC review rating from a human reviewer is used as an outcome label.

### Parameter Selection Related Features

In addition to the preprocessing features 530 (e.g. the log files), the other features that may be utilized as inputs into the machine learning model 540 may include at least one or more of the following features utilized in the example where parameter selection is utilized, rather than using standard MR parameters for data acquisition:
final cluster inclusion thresholds;
number of parameters sets in the largest cluster;
ratio of number of parameter sets in two largest clusters;
number of parameter sets in clusters size>1;
and others.

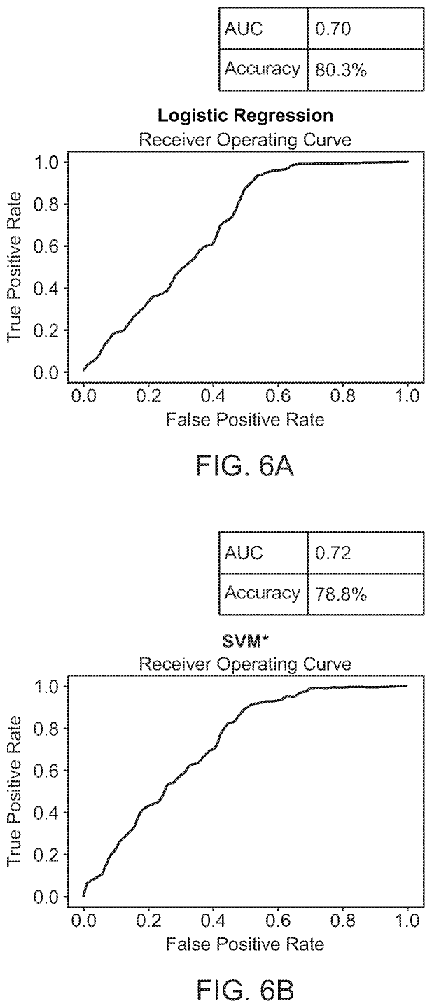

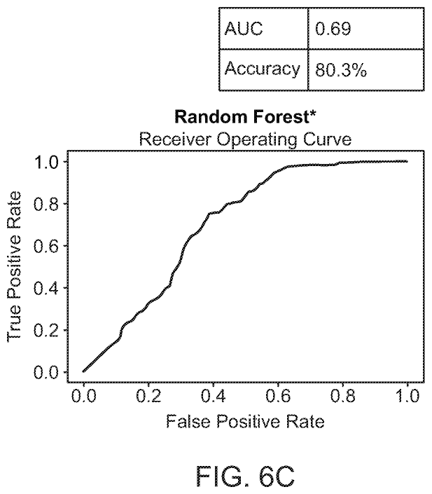

As shown in FIGS. 6A-6C, the disclosed technology for automated QC were tested on example data sets using parameter related features as inputs into the machine learning model. As illustrated, these models resulted in good accuracy (around 80 percent) in performing an automated QC function. In some examples, the combination of (i) identifying optimal parameters for pre-processing the MR images and (ii) using these parameters and related features as inputs into a machine learning algorithm to automatically pass or rejection MR images allows for reliable prediction of which images would pass manual QC. In some examples, the automated QC systems and methods were successfully applied to whole brain functional connectivity MRI data.

### MRI Preprocessing Features

In some examples, features generated by the Poldrack Lab at Stanford University software (MRIQC) may be utilized as inputs into the disclosed machine learning models. MRIQC is software developed by the Poldrack Lab at Stanford University. One of its features is the ability to generate measures of image quality from raw MR images. These Image Quality Metrics (IQMs) are used to predict manual QC labels on sMRI scans. The metrics are designated as “no-reference,” or having no ground-truth correct value. Instead, the metrics generated from one image can be judged in relation to a distribution of these measures over other sets of images, MRIQC generates IQMs from both structural and functional raw images.

The structural IQMs are divided into four categories: measures based on noise level, measures based on information theory, measures targeting specific artifacts, and measures not covered specifically by the other three. The functional IQMs are broken down into three categories: measures for spatial structure, measures for temporal structure, and measures for artifacts and others. In total there are 112 features generated by MRIQC, 68 structural features and 44 functional features. A full list of the features generated by MRIQC can be found at mriqc.readthedocs.io. The software can be run as either a Python library or Docker container. The present disclosure used the Docker version to generate IQMs on EMBARC and CNP.

### Log Files as Classifier Features

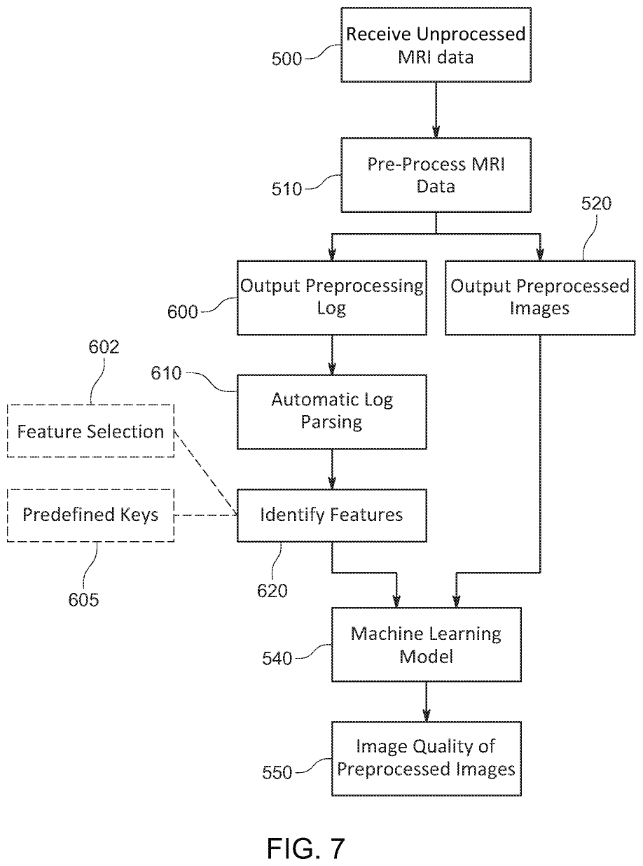

Referring now to FIG. 7, a flow chart is illustrated and shows another example of a method for predicting which images of a set of MR images will pass quality control. The method illustrated in FIG. 7 is the same as, or similar to, the method illustrated in FIG. 5, where the same reference numbers refer to the same elements.

At step 500, unprocessed MRI data is received. At step 510, the received MRI data is pre-processed. The pre-processed MRI data is then output as preprocessed images (step 520) and/or as a preprocessing log (step 600). After the preprocessing log is outputted (step 600), automatic log parsing is performed at step 610. The features can be identified at step 620, which can include feature selection (602) and/or predefined keys (605).

The preprocessed images (from step 520) and/or the identified features (from step 620) can be input into a machine learning model 540, which then outputs an image quality of the preprocessed images (step 550).

As such, various runtime logs output from the MRI preprocessing pipeline (e.g., the steps and elements shown in FIG. 7) were used as input features into the machine learning models (e.g., the machine learning model 540). MRI systems write events into log files while the system is running, including during preprocessing. In some examples, the features are derived from AFNI software comments run during an fMRI pre-processing pipeline. These commands are responsible for transforming the fMRI data into the final outputs that undergo manual QC. While an AFNI command (for instance) is executing, it outputs runtime logs.

In some examples, these runtime logs may be copied and saved into text files or other file types. These logs contain a large assortment of information, some of it pertaining to results of final or intermediate steps of a given command. The logs may include data relating to the cost or difference between the alignment of the structural and functional maps when preprocessing fMR images. These terminal command line logs can be predictive of how well the images are being preprocessed.

The log related fMRI features, in some examples, may be divided into four subgroups; Step Runtimes, Voxel Counts, Brain Coordinates, and Other Metrics. Step Runtime features quantify how long a given step, or set of steps, in the pipeline took to run. Voxel Count features measure the size of the output of a given step in the pipeline in terms of “voxels”, or volumetric 3D pixels. Brain Coordinate features simply refer to the X, Y, and Z coordinates of the bounding box of the brain image. Other Metrics are miscellaneous values that quantify the outcome of a certain step of the preprocessing pipeline.

An example of one of these Other Metrics is the cost function value associated with the step of the pipeline that aligns the structural and functional scans. In some examples, there could be 5, 10, 15, 20, 30, 35, 38, 42, or more log related features.

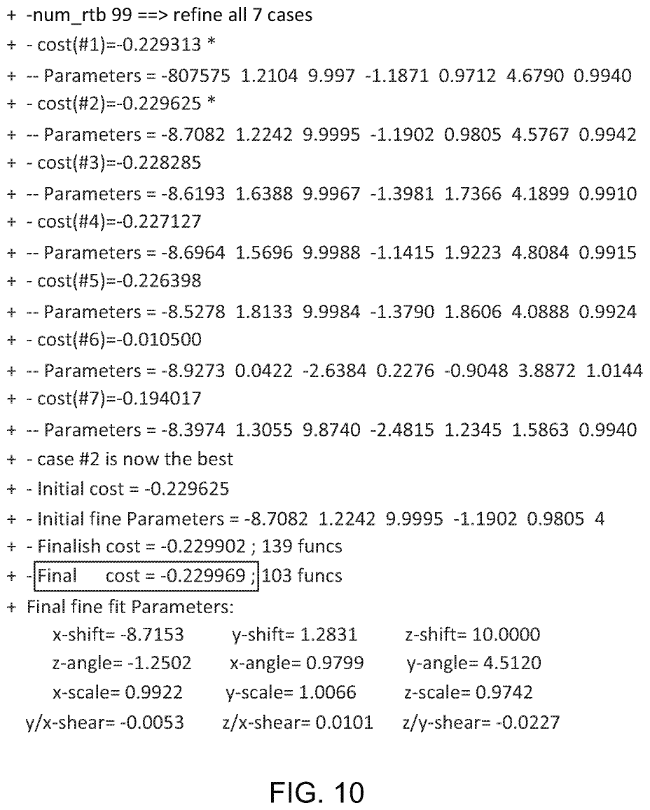

FIG. 10 illustrates an example of a runtime log text file output during preprocessing of a patient's fMRI scan (e.g., step 600 in FIG. 7). The highlighted portion is a feature identified as an input into the disclosed machine learning models.

### Automated Parsing and Feature Selection from Log Files

In some implementations, MR preprocessing log files may be automatically parsed (e.g. using a script using Python or a similar programming language) to identify features (e.g., steps 600-620 in FIG. 7). For example, a Python Regular Expression library can be used to parse the text files, and extract potentially informative features. In some implementations, this may include identification of all potential features (e.g., 620), and using a features selection procedure (e.g., 602) to identify the most relevant features from the log files. Accordingly, using these implementations, if the log files are textual based files such as .CSV, XLS, .DOC or other files, the technology could automatically search for numbers and adjacent text. The numbers could be entered into a database or other memory with references or tags to a category or descriptor that would be nearby text.

Then, various methods could be utilized to remove numbers that would not be good features, for instance by filtering for numbers with low variance between patients. Additionally, various feature selection methods associated with machine learning models may be utilized to identify the most important features by their textual tag (based on adjacent text).

For instance, a model-independent approach was applied. Specifically, a Hilbert-Schmidt Independence Criterion Lasso (HSIC Lasso) based feature selection can be used. HSIC Lasso utilizes a featurewise kernelized Lasso for capturing non-linear input-output dependency. A globally optimal solution can be efficiently calculated making this approach computationally inexpensive.

In the second phase, a model-dependent Forward Selection approach was applied. In some examples, a two-phase approach was chosen because it offers a good balance of classifier performance, fast computation, and generalization. The actual number of features selected depended on cross-validation performance.

Then, once a specific MR processing pipeline and its associated log files, have been fully processed to identify the best features, a machine learning model may be trained using those features. Accordingly, every new patient that is scanned using the same pipeline, the model could be utilized to process the log files associated with each image, and identify images likely to pass manual QC, for example.

### Experimental Testing of Classifiers

In some examples, data was used to test the disclosed log based approach. Specifically, an approach entitled “FMRI preprocessing Log mining for Automated, Generalizable Quality Control” (FLAG-QC) was used, in which features derived from mining runtime logs are used to train and as inputs into the classifier. The experimental data showed that classifiers trained on FLAG-QC features perform much better (AUC=0.79) than previously proposed feature sets (AUC=0.56), when testing their ability to generalize across studies.

To demonstrate the effectiveness of the disclosed technology, fMRI scans were used obtained from two separate studies: (1) Establishing Moderators and Biosignatures of Antidepressant Response for Clinical Care for Depression (EMBARC), (2) UCLA Consortium for Neuropsychiatric Phenomics LA5c (CNP). These data were utilized with different feature sets.

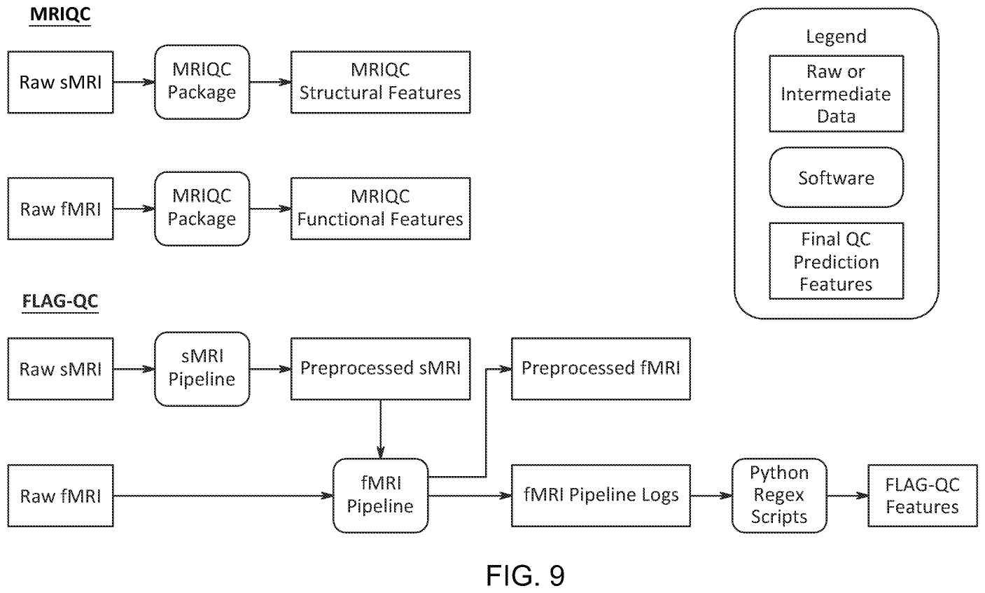

The features used to train QC classifiers come from two distinct pipelines: (1) FLAG-QC Features, a feature set novel to this study, and (2) MRIQC Features (e.g., those generated by the MRIQC software suite). A high-level block diagram showing the process for creating each set of features is shown in FIG. 9. The FLAG-QC and MRIQC features have been described herein.

### EMBARC

The EMBARC dataset was collected to examine a range of biomarkers in patients with depression to understand how they might be able to inform clinical treatment decisions. The study enrolled 336 patients aged 18-65, collecting demographic, behavioral, imaging, and wet biomarker measures for multiple visits over a period of 14 weeks. Data were acquired from the National Data Archive (NDA) repository on Jun. 19, 2018 with a license obtained by Blackthorn Therapeutics.

The disclosed study only analyzes data from sMRI and fMRI scans collected during patients' first and second visit to the study site. Specifically, T1-weighted structural MRI scans and T2*-weighted blood-oxygenation-level-dependent (BOLD) resting-state functional MRI scans were used, and were labelled as run 1. In total, 324 structural-functional MRI scan pairs were analyzed from the first site visit and 288 pairs from the second, producing a total of 612 scan pairs.

### CNP

The CNP dataset was collected to facilitate discovery of the genetic and environmental bases of variation in psychological and neural system phenotypes, to elucidate the mechanisms that link the human genome to complex psychological syndromes, and to foster breakthroughs in the development of novel treatments for neuropsychiatric disorders. The study enrolled a total of 272 participants aged 21-50. Within the participant group, there were 138 healthy individuals, 58 diagnosed with schizophrenia. 49 diagnosed with bipolar disorder, and 45 diagnosed with ADHD. All data were collected in a single visit per participant and included demographic, behavioral, and imaging measures.

Similar to EMBARC, data from participants that have both T1-weighted sMRI and T2*-weighted BOLD resting-state fMRI scans were used, and were labelled run 1. This amounts to 251 structural-functional MRI scan pairs.

Using both of these studies, it was demonstrated that the disclosed classifiers can accurately predict manual QC labels on fMRI scans within one data source using any of the feature sets mentioned above, but that only the log based feature set successfully generalized to data of another independent study. Data collected from the same study will be referred to as “within dataset” samples, while data collected from a study upon which a given model has not been trained will be referred to as “unseen study” data.

To predict fMRI QC labels, four different predictive models were evaluated using the sci-kit learn Python library: (1) Logistic Regression, (2) Support Vector Machines (SVM), (3) Random Forest, and (4) Gradient Boosting classifiers. The hyperparameters were tuned for the SVM, Random Forest, and Gradient Boosting models using 5-Fold Grid Search Cross Validation. Table 1 illustrates a summary of feature selection from “within dataset” and classification results.

TABLE 1
Summary of Within Dataset Ford Features
Selection Classification Results

| | EMBARC | | | CNP | | |
|---|---|---|---|---|---|---|
| | | # of | | | # of | |
| | Classifier | Features | | Classifier | Features | |
| Feature | w/Max | @ Max | MAX | w/Max | @ Max | MAX |
| Set | AUC | AUC | AUC | AUC | AUC | AUC |
| FLAG-QC | Random | 11 | 0.89 | SVM | 7 | 0.93 |
| | Forest | | | | | |
| MRIQC, | Logistic | 18 | 0.86 | SVM | 13 | 0.79 |
| Functional | Regression | | | | | |
| Structural | Gradient | 26 | 0.86 | Random | 10 | 0.85 |
| | Boosting | | | Forest | | |
| All | Random | 20 | 0.90 | SVM | 9 | 0.97 |
| Features | Forest | | | | | |

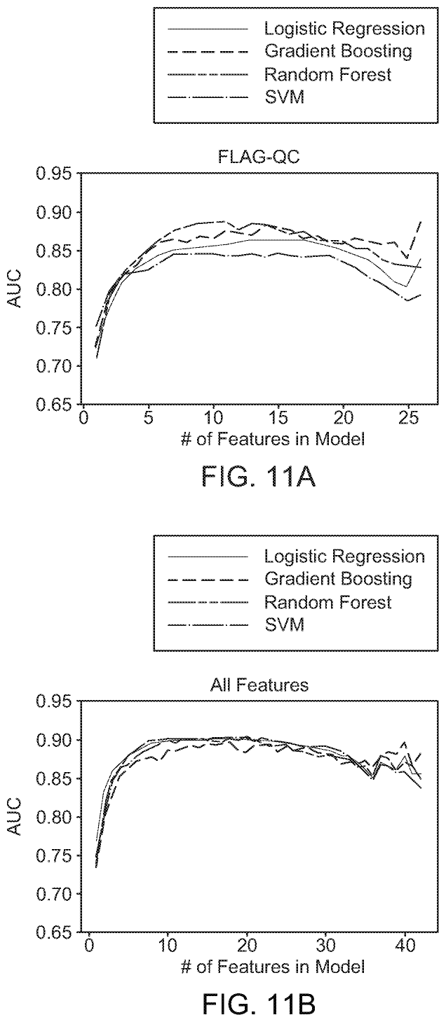

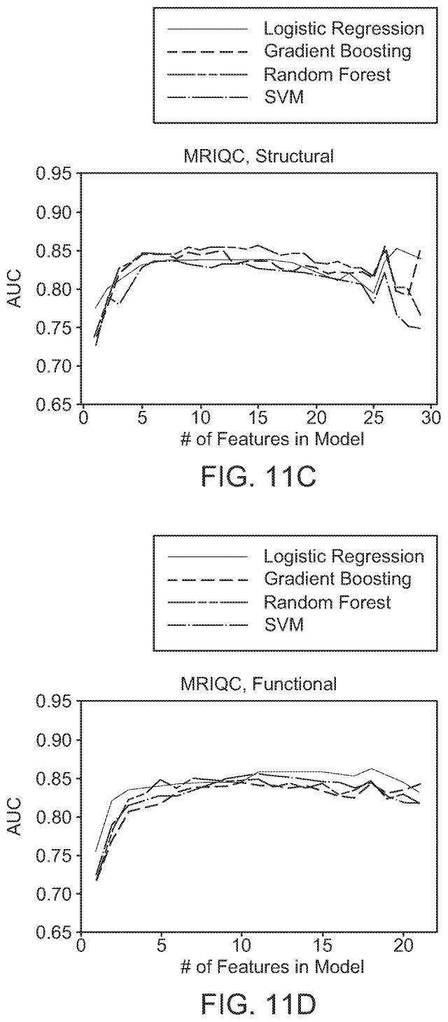

In some examples, manual QC labels were predicted for held out sets of scans within datasets collected in a single study. Logistic Regression, SVM, Random Forest, and Gradient Boosting classifiers were trained and tested separately on each of the three feature sets mentioned herein, labelled “FLAG-QC”, “MRIQC, Functional”, and “MRIQC, Structural”, as well as the ensemble of all, labelled “All Features”. To do so, each feature-model pair was evaluated within a 5-fold Cross Validation scheme, first using HSIC Lasso to reduce the dimensionality of the feature space. Then, Forward Feature Selection was run, and the mean AUC across folds for each number of selected features were reported. The results using these methods for the EMBARC dataset are shown in FIGS. 11A-11D, and summary results for both EMBARC and CNP datasets are displayed in Table 1.

In the EMBARC dataset, after Forward Feature Selection, it was found that the FLAG-QC feature set achieves an AUC of 0.89, The other individual feature sets perform slightly worse with the AUC being 0.86 for the MRIQC, Functional features and 0.86 for the MRIQC, Structural features. It was also observed that by using all of the features together creates the classifier with the best performance, achieving an AUC of 0.90. However, it was observed that there was variability in which model performed best on each feature set, all models performed reasonably well across all feature sets, with the lowest feature-model AUC being 0.83 (MRIQC, Structural—SVM).

The same procedure was replicated on the CNP dataset, resulting in an AUC of 0.93 for the FLAG—QC (SVM); 0.79 for the MRIQC, Functional Features (SVM); 0.85 for MRIQC, Structural (Random Forest); and 0.97 for the ensemble feature sets (SVM). A similar pattern was seen with the FLAG-QC features outperform the MRIQC feature sets (though by a larger magnitude this time), and the combination of all feature sets outperforms any individual set. Also again all feature-model pairs perform reasonably accurately (min AUC 0.77 with MRIQC, Functional features using Gradient Boosting).

### Unseen Study Dataset as Test Set

The same modeling framework was also applied to predict QC labels on one dataset, while the classifier was trained on data collected from a completely separate study. In this example, all 612 labelled scans from the EMBARC dataset were used as the training set. Accordingly, the results from EMBARC was used within dataset cross validation prediction with Forward Feature Selection to select the model that will be evaluated on the test set, CNP. For each feature set, the classifier with the highest AUC was selected. The classifiers selected for each feature set are shown in Table 1.

Within each feature set, it was again started by running HSIC Lasso on the training dataset for an initial model independent feature selection, and then performing Forward Feature Selection to choose the final set of features tested on CNP data. Finally, one last 5-fold CV parameter grid search was performed to tune and train the model specifically for the final selected set of features. Using this framework, manual QC labels on scans from the CNP dataset were predicted to evaluate the model's performance.

The FLAG-QC features performed much better when predicting on the unseen study data from the CNP dataset than any other set of features, attaining an AUC of 0.79 as seen in Table 2.

TABLE 2
Classifier Metrics

| | | MRIQC | MRIQC | |
|---|---|---|---|---|
| Metric | FLAG-QC Logs | Functional | Structural | All Features |
| AUC | 0.79 | 0.56 | 0.56 | 0.64 |
| Accuracy | 74.90% | 61.35% | 56.57% | 64.54.% |
| Precision | 0.72 | 0.62 | 0.59 | 0.64 |
| Recall | 0.95 | 0.85 | 0.83 | 0.93 |

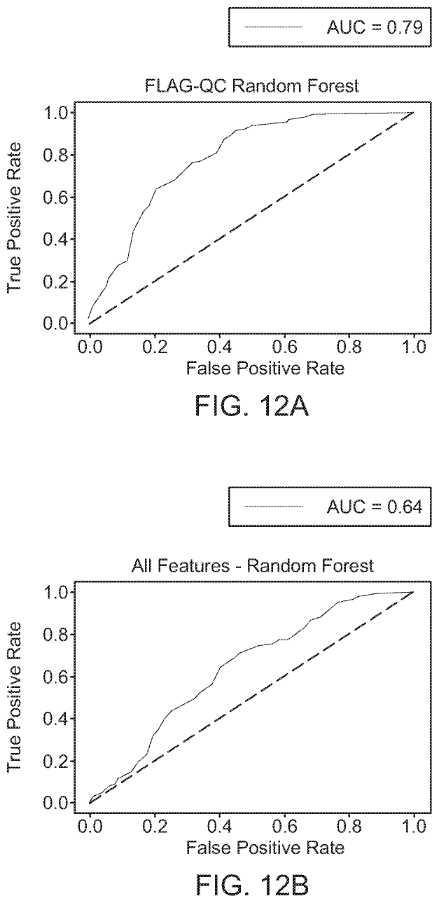

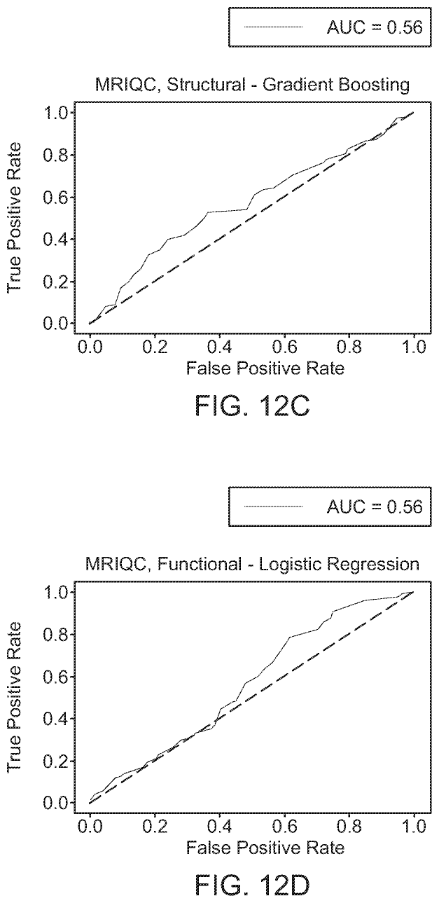

The ROC curves from these predictions shown in FIGS. 12A-12D and Table 2 clearly display a difference in performance between the novel feature set of the present disclosure and those previously proposed. The individual MRIQC feature sets perform much worse on the unseen study, each only reaching an AIX of 0.56. Additionally, the second best performing set of features is “All Features” with an AUC of 0.64. This set of course contains the FLAG-QC features, further highlighting the importance of the FLAG-QC features in the classifiers' ability to generalize across datasets. The pronounced drop in performance in unseen study prediction associated with all models that include MRIQC features implies that these features may lead to greater overfitting on the training set as compared to that of FLAG-QC. The results achieved using the FLAG-QC features demonstrates the disclosed classifier's generalizability in predicting fMRI QC labels in unseen studies.

### Additional Implementations

According to some implementations of the present disclosure, a method of analyzing MRI data includes receiving unprocessed MRI data corresponding to a set of MR images of a biological structure. The received MRI data is preprocessed, wherein the preprocessing includes (i) performing, for each MR image in the set of MR images, a structural-functional alignment, (ii) performing a skull-stripping procedure, and (iii) outputting a plurality of parameter sets related to the preprocessing. A plurality of functional connectivity matrices is generated based on the plurality of parameter sets. Similar matrices in the plurality of functional connectivity matrices are identified to yield a plurality of matrix clusters. A dominant cluster of the plurality of matrix clusters is selected. A subset of parameters of the plurality of parameter sets corresponding to the dominant matrix is outputted.

In some implementations, identifying similar matrices further includes determining a Frobenius norm of a pairwise difference between matrices in the plurality of functional connectivity matrices. Matrices in the plurality of functional connectivity matrices are grouped into a subset cluster when the determined Frobenius norm is less than a threshold value. The subset cluster is outputted into the plurality of matrix clusters. In some such implementations, identifying similar matrices further includes increasing the threshold value until a size of a largest cluster in the plurality of matrix clusters is twice as large as a size of a next-largest cluster in the plurality of matrix clusters.

In some implementations, the plurality of parameter sets corresponds to four parameters from a plurality of parameters associated with at least one of: the structural-functional alignment and skull-stripping procedure.

In some implementations, the output subset of parameters corresponds to a centroid of the dominant cluster.

In some implementations, the received MRI data with the output subset of parameters is processed to yield a set of processed MR images.

In some implementations, the received MRI data corresponds to MRI data for a subject.

In some implementations, a brain of a subject is scanned to output the set of MR images.

According to some implementations of the present disclosure, a system for analyzing MRI data includes a memory, and a control system. The memory contains machine readable medium, which includes machine executable code having stored thereon instructions for performing a method. The control system is coupled to the memory, and includes one or more processors. The control system is configured to execute the machine executable code to cause the control system to receive unprocessed MRI data corresponding to a set of MR images of a biological structure. The received MRI data is preprocessed, wherein preprocessing includes (i) performing, for each MR image in the set of MR images, a structural-functional alignment, (ii) performing a skull-stripping procedure, and (iii) outputting a plurality of parameter sets related to the preprocessing. A plurality of functional connectivity matrices is generated based on the plurality of parameter sets. Similar matrices in the plurality of functional connectivity matrices are identified to yield a plurality of matrix clusters. A dominant cluster of the plurality of matrix clusters is selected. A subset of parameters of the plurality of parameter sets corresponding to the dominant matrix is outputted.

According to some implementations of the present disclosure, a non-transitory machine-readable medium stores thereon instructions for performing a method. The non-transitory machine-readable medium includes machine executable code, which when executed by at least one machine causes the machine to receive unprocessed MRI data corresponding to a set of MR images of a biological structure. The received MRI data is preprocessed, wherein preprocessing includes (i) performing, for each MR image in the set of MR images, a structural-functional alignment, (ii) performing a skull-stripping procedure, and (iii) outputting a plurality of parameter sets related to the preprocessing. A plurality of functional connectivity matrices is generated based on the plurality of parameter sets. Similar matrices in the plurality of functional connectivity matrices are identified to yield a plurality of matrix clusters. A dominant cluster of the plurality of matrix clusters is selected. A subset of parameters of the plurality of parameter sets corresponding to the dominant matrix is outputted.

According to sonic implementations of the present disclosure, a system for analyzing MRI data includes a memory, and a control system. The memory contains machine readable medium, which includes machine executable code having stored thereon instructions for performing a method. The control system is coupled to the memory, and includes one or more processors. The control system is configured to execute the machine executable code to cause the control system to receive unprocessed MRI data corresponding to a set of MR images of a biological structure. The received MRI data is preprocessed, wherein preprocessing includes (i) performing, for each MR image in the set of MR images, a structural-functional alignment, (ii) performing a skull-stripping procedure, and (iii) outputting a plurality of parameter sets related to the preprocessing. A plurality of whole brain functional connectivity matrices is generated based on the plurality of parameter sets. Similar matrices in the plurality of whole brain functional connectivity matrices are identified to yield a plurality of matrix clusters. A dominant cluster of the plurality of matrix clusters is selected. A subset of parameters of the plurality of parameter sets corresponding to the dominant cluster is outputted. Using a machine learning model, a set of features associated with the set of MR images based on the subset of parameters is processed to determine a subset of the set of MR images that are predicted to pass quality control.

In some implementations, the machine learning model includes a logistic regression, support vector machine, a random forest model, or any combination thereof.

In some implementations, the set of features includes a final cluster inclusion threshold, a number of parameters sets in a largest cluster, a ratio of number of parameter sets in the largest cluster and a second largest cluster, a number of parameter sets in which a cluster size is great than 1, or any combination thereof.

In some implementations, the machine learning model is trained using outcome labels based on manual QC ratings.

In some implementations, the set of features includes a set of data from MRI preprocessing runtime logs.

In some implementations, the control system is further configured to process additionally received unprocessed MRI data with the output subset of parameters to yield a set of processed MR images.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices)

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### REFERENCES

Alfaro-Almagro, F.; et al. 2018. Image processing and quality control for the first 10,000 brain imaging datasets from uk biobank. NeuroImage 166:400-424.

Casey, B.; et al. 2018. The adolescent brain cognitive development (abcd) study: Imaging acquisition across 21 sites. Developmental Cognitive Neuroscience 32:43-54. The Adolescent Brain Cognitive Development (ABCD) Consortium: Rationale, Aims, and Assessment Strategy.

Cox, R. W. 1996. Afni: Software for analysis and visualization of functional magnetic resonance neuroimages. Computers and Biomedical Research 29(3):162-173.

Cremers, R.; Wager, T. D.; and Yarkoni, T. 2017. The relation between statistical power and inference in fmri. PLOS ONE 12(11):1-20.

Di Martino, A.; et al. 2014. The autism brain imaging data exchange: towards a large-scale evaluation of the intrinsic brain architecture in autism. Molecular Psychiatry 19(6):659-667.

Drysdale, A. T.; et al. 2016. Resting-state connectivity biomarkers define neurophysiological subtypes of depression. Nature Medicine 23:28 EP Article.

Essen, D. V.; et al. 2012. The human connectome project: A data acquisition perspective. NeuroImage 62(4):2222-2231. Connectivity.

Esteban. O.; et al. 2017, Mriqc: Advancing the automatic prediction of image quality in mri from unseen sites. PLOS ONE 12(9):1-21.

Fischl, B.; et al. 2002. Whole brain segmentation: Auto-mated labeling of neuroanatomical structures in the human brain. Neuron 33(3):341-355.

Gao, S.; Calhoun, V. D.; and Sui, J. 2018. Machine learning in major depression: From classification to treatment outcome prediction. CNS Neuroscience & Therapeutics 24(11): 1037-1052.

Hastie, T.; Tibshirani, R.; and Friedman, J. 2009. The Elements of Statistical Learning: Data Mining, Inference, and Prediction. Springer.

Liu, Y.; et al. 2018. Highly predictive transdiagnostic features shared across schizophrenia, bipolar disorder, and adhd identified using a machine learning based approach. bioRxiv.

Liu, Y.; et al. 2019. Machine learning identifies large-scale reward-related activity modulated by dopaminergic enhancement in major depression. Biological Psychiatry: Cognitive Neuroscience and Neuroimaging.

Lu, W.; Dong, K.; Cui, D.; Jiao, Q; and Qiu, J. 2019. Quality assurance of human functional magnetic resonance imaging: a literature review. Quantitative Imaging in Medicine and Surgery 9(6).

Mellem, M. S.; et al. 2018. Machine learning models identify multimodal measurements highly predictive of transdiagnostic symptom severity for mood, anhedonia, and anxiety. bioRxiv.

Mortamet, B.; et al. 2009. Automatic quality assessment in structural brain magnetic resonance imaging. Magnetic Resonance in Medicine 62(2):365-372.

Pizarro, R. A.; et al. 2016. Automated quality assessment of structural magnetic resonance brain images based on a supervised machine learning algorithm. Frontiers in Neuroinformatics 10:52.

Poldrack, R. A.; et al. 2016. A phenome-wide examination of neural and cognitive function. Scientific Data 3(1):160110.

Reuter, M. 2013. Freesurfer.

Soares, J. M.; et al, 2016, A hitchhiker's guide to functional magnetic resonance imaging. Frontiers in Neuroscience 10:515.

Trivedi, M. H.; et al. 2016. Establishing moderators and biosignatures of antidepressant response in clinical care (embarc): Rationale and design. Journal of Psychiatric Research 78:11-23.

Woodard, J. P., and Carley-Spencer, M. P. 2006.

No-reference image quality metrics for structural mri. Neuroinformatics 4(3):243-262.

Yamada, M.; Jitkrittum, W.; Sigal, L.; Xing, E. P.; and Sugiyama, M. 2014. High-dimensional feature selection by feature-wise kernelized lasso. Neural Computation 26(1): 185207.

### CONCLUSION

The various methods and techniques described above provide a number of ways to carry out the invention. Of course, it is to be understood that not necessarily all objectives or advantages described can be achieved in accordance with any particular implementation described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some implementations specifically include one, another, or several features, while others specifically exclude one, another, or several features, while still others mitigate a particular feature by inclusion of one, another, or several advantageous features.

Furthermore, the skilled artisan will recognize the applicability of various features from different implementations. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse implementations.

Although the application has been disclosed in the context of certain implementations and examples, it will be understood by those skilled in the art that the implementations of the application extend beyond the specifically disclosed implementations to other alternative implementations and/or uses and modifications and equivalents thereof.

In some implementations, the terms “a” and “an” and “the” and similar references used in the context of describing a particular implementation of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise dearly contradicted by context. The use of any and all examples, or exemplary language (for example, “such as”) provided with respect to certain implementations herein is intended merely to better illuminate the application and does not pose a limitation on the scope of the application otherwise claimed. No language in the specification should be construed as indicating any non-claimed element essential to the practice of the application.

Certain implementations of this application are described herein. Variations on those implementations will become apparent to those of ordinary skill in the art upon reading the foregoing description. It is contemplated that skilled artisans can employ such variations as appropriate, and the application can be practiced otherwise than specifically described herein. Accordingly, many implementations of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the implementations of the application disclosed herein are illustrative of the principles of the implementations of the application. Other modifications that can be employed can be within the scope of the application. Thus, by way of example, but not of limitation, alternative configurations of the implementations of the application can be utilized in accordance with the teachings herein. Accordingly, implementations of the present application are not limited to that precisely as shown and described.

What is claimed is:

1. A system for analyzing MRI data, the system comprising:
a memory containing machine readable medium including machine executable code having stored thereon instructions for performing a method; and
a control system coupled to the memory and having one or more processors, the control system configured to execute the machine executable code to cause the control system to:
receive unprocessed MRI data corresponding to a set of MR images;
perform a preprocessing on the received unprocessed MRI data to output a preprocessed set of MR images.
output a set of features related to the preprocessing; and
process, using a machine learning model, the set of features to determine a subset of the preprocessed set of MR images that have a threshold image quality.

2. The system of claim 1, wherein the threshold image quality includes an image quality sufficient to pass manual quality control.

3. The system of claim 1, wherein the threshold image quality includes an image quality suitable for further processing by a model to identify a set of functional Magnetic Resonance Imaging (fMRI) features.

4. The system of claim 3, wherein the set of fMRI features includes at least functional connectivity.

5. The system of claim 1, wherein the preprocessing includes performing, for each MR image in the set of MR images, a structural-functional alignment.

6. The system of claim 1, wherein the machine learning model includes a logistic regression model, a support vector machine, a gradient boosting machine, or a random forest model.

7. The system of claim 1, wherein the machine learning model is trained using outcome labels based on manual QC ratings.

8. The system of claim 1, wherein the set of features includes a set of log data from MRI preprocessing runtime logs.

9. The system of claim 8, wherein the set of log data from MRI preprocessing runtime logs includes data in text format relating to a quantitative assessment of structural-functional alignment.

10. The system of claim 8, wherein the set of log data from MRI preprocessing runtime logs includes at least one of: preprocessing step runtimes, brain coordinates, structural-functional alignment cost values, a quantity of edits made to the set of MR images, and an angle of image capture of the brain in the set of MR images.

11. The system of claim 1, wherein the control system is further configured to store the subset of the set of MR images in the memory.

12. The system of claim 1, wherein the preprocessing further includes a skull stripping procedure.

13. The system of claim 1, wherein the preprocessed set of MR images includes structural MR images.

14. The system of claim 1, wherein the preprocessed set of: MR images includes functional MR images.

15. The system of claim 1, wherein the set of MR images includes unprocessed functional MRI data and unprocessed structural MRI data representing a brain for each patient.

16. A method for analyzing MRI data, the method comprising:
receiving unprocessed MRI data corresponding to a set of MR images;
performing a preprocessing on the received unprocessed MRI data to output a preprocessed set of MR images.
outputting a set of features related to the preprocessing; and
processing, using a machine learning model, the set of features to determine a subset of the preprocessed set of MR images that have a threshold image quality.

17. The method of claim 16, wherein the threshold image quality includes an image quality suitable for further processing by a model to identify a set of functional Magnetic Resonance Imaging (fMRI) features.

18. The method of claim 16, wherein the set of features includes a set of log data from MRI preprocessing runtime logs.

19. The method of claim 18, wherein the set of log data from MRI preprocessing runtime logs includes data in text format relating to a quantitative assessment of structural-functional alignment.

20. The method of claim 18, wherein the set of log data from MRI preprocessing runtime logs includes at least one of: preprocessing step runtimes, brain coordinates, structural-functional alignment cost values, a quantity of edits made to the set of MR images, and an angle of image capture of the brain in the set of MR images.

21. A non-transitory machine-readable medium having stored thereon instructions for performing a method, the non-transitory machine-readable medium including machine executable code which when executed by at least one machine, causes the machine to:
receive unprocessed MRI data corresponding to a set of MR images;
perform a preprocessing on the received unprocessed MRI data to output a preprocessed set of MR images.
output a set of features related to the preprocessing; and
process, using a machine learning model, the set of features to determine a subset of the preprocessed set of MR images that have a threshold image quality.

22. The non-transitory machine-readable medium of claim 21, wherein the set of features includes a set of log data from MRI preprocessing runtime logs.

23. The non-transitory machine-readable medium of claim 22, wherein the set of log data from MRI preprocessing runtime logs includes data in text format relating to a quantitative assessment of structural-functional alignment.

24. The non-transitory machine-readable medium of claim 22, wherein the set of log data from MRI preprocessing runtime logs includes at least one of: preprocessing step runtimes, brain coordinates, structural-functional alignment cost values, a quantity of edits made to the set of MR images, and an angle of image capture of the brain in the set of MR images.

25. The non-transitory machine-readable medium of claim 21, wherein the preprocessing further includes a skull stripping procedure.
